(19) United States
(12) Patent Application Publication
(10) Pub. No.: US 2026/0146130 A1
(43) Pub. Date: May 28, 2026

(54) POLYMER DOT, PREPARATION METHOD AND USE THEREOF

(71) Applicant: China Medical University, Taichung City (TW)

(72) Inventors: Tzong-Yuan Juang, Taichung City (TW); Chang-Cheng Chang, Taichung City (TW); Jia-Qi Chen, Taichung City (TW); Ching-Hsuan Lin, Taichung City (TW); Wen-Ping Jiang, Taipei City (TW)

(73) Assignee: China Medical University, Taichung City (TW)

(21) Appl. No.: 19/398,086

(22) Filed: Nov. 24, 2025

(30) Foreign Application Priority Data
Nov. 22, 2024 (TW) 113144991

(51) Int. Cl. C08G 73/10; A61K 49/00; A61P 31/04; C08J 3/24

(52) U.S. Cl. CPC C08G 73/105; A61K 49/0019; A61P 31/04; C08J 3/247; C08J 2379/08

(57) ABSTRACT

A polymer dot includes a nitrogen-containing carbon dot that is free of benzene rings. A preparation method of the polymer dot includes: treating an A₂ type monomer and a Bs type monomer by a one-pot synthesis to obtain the polymer dot. A use of the polymer dot is for manufacture of at least one of anti-inflammatory, antibacterial, and antioxidant compositions.

### BACKGROUND OF THE INVENTION

### 1. Field of the Invention

The present disclosure relates to a polymer dot, and more particularly to a polymer dot comprising a nitrogen-containing carbon dot that is free of benzene rings, and a method for preparing the same, and use thereof.

### 2. Description of the Prior Art

Diseases caused by oxidative stress pose a significant challenge to human health. However, antioxidant drugs capable of reducing oxidative stress and preventing related diseases remain limited.

Natural enzymes derived from natural substances are known antioxidant agents. Nevertheless, they suffer from several drawbacks, including high production costs, low yields, structural instability, complex compositions, and large molecular sizes that limit cellular permeability, which severely restricts their clinical applications.

Accordingly, there remains an urgent need in the art for materials and preparation methods capable of addressing the foregoing issues.

### SUMMARY OF THE INVENTION

To address the foregoing issues, the present disclosure provides a polymer dot comprising a nitrogen-containing carbon dot that is free of benzene rings.

The present disclosure further provides a method for preparing a polymer dot, comprising subjecting an A2-type monomer and a B3-type monomer by a one-pot synthesis to form a nitrogen-containing carbon dot.

The present disclosure also provides a use of the polymer dot in a preparation of a composition for at least one of anti-inflammatory, antibacterial, and/or antioxidant purposes.

In at least one embodiment of the present disclosure, polymer dots in the nanoscale range can be produced via a simple, low-cost, and facile preparation method. These polymer dots exhibit photostability, low cytotoxicity, biocompatibility, nitric oxide scavenging ability, antibacterial properties, and excellent antioxidant activity, and may be formulated into compositions for anti-inflammatory, antibacterial, and/or antioxidant applications.

### BRIEF DESCRIPTION OF THE DRAWINGS

The patent or application file contains at least one drawing executed in color. Copies of this patent or patent application publication with color drawings will be provided by the Office upon request and payment of the necessary fee. The following drawings form part of the present specification and are included to further demonstrate certain aspects of the present disclosure. The present disclosure may be better understood by reference to one or more of these drawings in combination with the detailed description of specific embodiments presented herein.

FIG. 1 is a fluorescence image and spectrum diagram according to at least one embodiment of the present disclosure, showing the fluorescence performance of the polymer dots.

FIG. 2A is a transmission electron microscopy (TEM) image according to at least one embodiment of the present disclosure, showing the morphology and particle size of the polymer dots and/or their clusters.

FIG. 2B is a schematic diagram of the average particle size distribution of the polymer dots according to at least one embodiment of the present disclosure.

FIG. 3A is a schematic diagram showing the main elemental component and content of the polymer dots according to at least one embodiment of the present disclosure.

FIG. 3B is a schematic diagram showing the bonding structure of the polymer dots according to at least one embodiment of the present disclosure.

FIG. 4 is a schematic diagram of the diffraction peaks of the polymer dots according to at least one embodiment of the present disclosure.

FIG. 5A is a schematic diagram showing the effect of the polymer dots on the cell viability of mouse macrophages RAW 264.7 according to at least one embodiment of the present disclosure.

FIG. 5B is a schematic diagram showing the effect of the polymer dots on the cell viability of RAW 264.7 cells induced by lipopolysaccharide (LPS)-induced inflammation according to at least one embodiment of the present disclosure. LPS: lipopolysaccharide; PD: polymer dot.

FIG. 5C is a schematic diagram showing the effect of the polymer dots on the cell viability of human hepatocellular carcinoma cells (HepG2) according to at least one embodiment of the present disclosure.

FIG. 6A is a schematic diagram showing the nitric oxide (NO) scavenging ability of the polymer dots in RAW 264.7 cells according to at least one embodiment of the present disclosure.

FIG. 6B is a schematic diagram showing the nitric oxide (NO) scavenging ability of the polymer dots in RAW 264.7 cells induced by LPS-induced inflammation according to at least one embodiment of the present disclosure. LPS: lipopolysaccharide; PD: polymer dot. *P<0.05 and **P<0.01 (compared with LPS); ###P<0.001 (compared with control).

FIG. 7 is an image showing the antibacterial activity of the polymer dots according to at least one embodiment of the present disclosure.

FIG. 8A is a schematic diagram showing the DPPH (1-diphenyl-2-picrylhydrazyl) scavenging ability of the polymer dots according to at least one embodiment of the present disclosure. **P<0.01 (compared with preparation example).

FIG. 8B is a schematic diagram showing the ABTS [2,2′-Azino-bis(3-ethylbenzothiazoline-6-sulfonic acid)] scavenging ability of the polymer dots according to at least one embodiment of the present disclosure.

### DETAILED DESCRIPTION

The embodiments of the present disclosure are further illustrated by the following examples. A person having ordinary skill in the art can readily understand the spirit, advantages, and effects of the present disclosure based on the content described herein. However, the specific embodiments disclosed herein are not intended to limit the present disclosure. The present disclosure may also be implemented or applied in other different ways. The various details set forth herein may be modified or adapted from different perspectives and for different applications without departing from the spirit of the present disclosure.

The proportions, structures, sizes, and other features shown in the accompanying drawings are provided solely to aid in understanding the technical content disclosed herein and are not intended to limit the scope of implementation of the present disclosure. Therefore, any change in proportions, structural modifications, or size adjustments that do not materially affect the intended purpose or effect of the present disclosure shall fall within the scope of the technical content disclosed herein.

As used herein, the terms “comprise,” “comprises,” “include,” “includes,” “having,” and similar expressions are intended to indicate the presence of stated features, integers, steps, components, or combinations thereof, but do not preclude the presence or addition of one or more other features, integers, steps, components, or groups thereof, unless otherwise specified.

Unless expressly stated otherwise, the singular forms “a,” “an,” and “the” as used herein shall be interpreted to include the plural forms as well. Additionally, the terms “or” and “/” as used herein are intended to be used interchangeably with “and/or” unless otherwise specified.

As used herein, the term “about” refers to values within typical tolerances in the art. For example, “about” may refer to a value within approximately two standard deviations of the mean. When “about” precedes a series of values or a range, it is understood to modify each of the values in the series or range. For instance, numerical values are intended to encompass variations of ±25%, ±20%, ±10%, ±5%, ±1%, ±0.5%, ±0.2%, or ±0.1%. Such variations may result from experimental error, variability in the measurement or processing of compounds, compositions, concentrates, or formulations, differences in the source, manufacture, or purity of starting materials or ingredients used, or similar considerations.

The numerical ranges disclosed herein are inclusive and combinable. Any value within a disclosed range may serve as an upper or lower endpoint to define a sub-range. For example, the range “−1 mV to 20 mV” is understood to include any sub-range such as −0.5 mV to 19.5 mV, 0 mV to 19 mV, or 0.5 mV to 18.5 mV. Additionally, multiple numerical endpoints disclosed herein may be used interchangeably as upper or lower limits to create new ranges; for example, −1 mV, 0 mV, and 20 mV may define ranges such as −1 mV to 0 mV, 0 mV to 20 mV, or −1 mV to 20 mV.

As used herein, the term “polymer dot” refers to a nanoscale polymer formed by chemical crosslinking of molecules under thermal conditions. The particle size of the polymer dot is within the nanometer scale in aqueous environments. Unless otherwise specified, the terms “carbon dot” and “carbon nanodot (CND)” are used interchangeably. Carbon nanodots, due to their optical properties such as resistance to fluorescence quenching and photobleaching, along with antimicrobial activity, making them suitable for applications in materials science and biomedical fields.

Unless otherwise specified, the terms “dendritic” and “hyperbranched” are used interchangeably herein.

As used herein, the term “oxidative stress” refers to a state of imbalance caused by excessive reactive oxygen species (ROS) or free radicals generated during metabolic processes, which overwhelms the body's antioxidant defense system. In some embodiments, the polymer dots of the present disclosure exhibit excellent free radical scavenging capabilities and may serve as effective novel antioxidants, thereby contributing to anti-aging effects and lifespan extension.

As used herein, the term “nanozyme” refers to nanoparticles exhibiting enzyme-like catalytic activity and may serve as alternatives to natural enzymes. Nanozymes may be used to treat diseases such as sepsis, Alzheimer's disease, oral ulcers, cardiovascular diseases, COVID-19, inflammation, or tissue injury, although the present disclosure is not limited thereto. Nanozymes are a novel class of enzyme-mimicking nanomaterials that possess greater stability and tunable catalytic activity compared with natural enzymes. In some embodiments, the polymer dots of the present disclosure are synthesized by gram-scale processes (e.g., one-pot synthesis, or a combination of one-pot synthesis and hydrothermal method) and exhibit bioactivity attributable to their nitrogen-containing structures, particularly antioxidant activity that mimics natural enzymes (enzyme-like activity). Accordingly, the polymer dots of the present disclosure can function as nanozymes and serve as materials for delaying aging and treating diseases caused by oxidative stress.

As used herein, the term “atypical fluorescent polymer” refers to polymers comprising non-conjugated aliphatic chromophores containing nitrogen (N), oxygen (O), sulfur(S), and phosphorus (P) atoms, as well as triple or double bonds (e.g., C═O or C≡N).

As used herein, the term “atypical fluorescent polymer dot” refers to a wholly aliphatic polymer dot having a non-conjugated hyperbranched structure and prepared from an atypical fluorescent polymer.

As used herein, the term “wholly aliphatic” refers to being free of benzene rings.

As used herein, the term “cluster” refers to an aggregate formed by crosslinking of the polymer dots, the structure of which is compact and exhibits spectral characteristics distinct from those of other polymer dots, thereby possessing unique optical properties.

As used herein, the term “crosslink-enhanced emission (CEE) effect” refers to the luminescence mechanism of the polymer dot clusters, in which fluorescence characteristics are induced or amplified through polymer chemical crosslinking.

As used herein, the term “d(002)” refers to the interplanar spacing of a crystal plane of the polymer dot corresponding to the graphite (002) plane.

As used herein, the term “d(100)” refers to the interplanar spacing of a crystal plane of the polymer dot corresponding to the graphite (100) plane.

As used herein, the term “at %” refers to atomic percentage (atomic %).

As used herein, the term “subject” refers to an individual, cell, or microorganism. In at least one embodiment of the present disclosure, the cell may be a macrophage or a cancer cell, but the present disclosure is not limited thereto. In at least one embodiment of the present disclosure, the microorganism may be Escherichia coli, but the present disclosure is not limited thereto.

As used herein, the term “individual” refers to any vertebrate, including but not limited to humans, monkeys, mice, rats, marmots, ferrets, rabbits, hamsters, cattle, horses, pigs, deer, dogs, cats, foxes, wolves, chickens, emus, or ostriches. In at least one embodiment of the present disclosure, the individual is a mammal, such as a primate (e.g., human, chimpanzee, gorilla, orangutan, ape, or monkey) or a rodent (e.g., mouse, rat, marmot, or hamster).

As used herein, the term “effective amount” refers to an amount of an active component (e.g., a polymer dot) or a composition containing such active component that is sufficient to produce a desired effect (e.g., antioxidant, anti-inflammatory, or antibacterial) in a subject in need thereof. In some embodiments, an effective amount prevents or reduces the appearance and/or symptoms associated with an undesired condition. The effective amount may vary as determined by one of ordinary skill in the art depending on the excipients used, route of administration, combination with other therapies, or the condition to be treated, without limitation.

As used herein, the term “administering” refers to a technique by which an active component (e.g., a polymer dot) or a composition comprising such an active component is introduced into a subject via a method or route such that at least a portion of the active component is localized at a desired site to exert a desired effect. In some embodiments, the active component of the present disclosure may be administered to a local or systemic site of the subject via oral administration, parenteral administration, injection, subcutaneous administration, sublingual administration, or topical administration, among others. However, the present disclosure is not limited thereto.

As used herein, the term “pharmaceutically or cosmetically acceptable carrier” refers to a material, medium, or composition that is pharmaceutically or cosmetically acceptable. Non-limiting examples include solid or liquid fillers, binders, diluents, preservatives, biocompatible solvents, disintegrants, lubricants, suspending agents, flavoring agents, capsule materials, thickeners, acids, surfactants, chelating agents, wetting agents, or any combinations thereof. In some embodiments, each component is “pharmaceutically or cosmetically acceptable,” meaning that it is compatible with the other components of the cosmetic or pharmaceutical formulation and suitable for contact with organs or tissues of a subject (e.g., mammals) without causing significant toxicity, allergic reactions, irritation, immunogenicity, or other complications or problems. Reference may be made to the following: Remington: The Science and Practice of Pharmacy, 22nd ed., Allen Ed., Philadelphia, PA, 2012; Handbook of Pharmaceutical Excipients, 7th ed., Rowe et al., Eds., The Pharmaceutical Press and the American Pharmaceutical Association, 2012; Handbook of Pharmaceutical Additives, 3rd ed., Ash and Ash Eds., Gower Publishing Company, 2007; Pharmaceutical Preformulation and Formulation, 2nd ed., Gibson Ed., CRC Press LLC, Boca Raton, FL, 2009.

In at least one embodiment of the present disclosure, an X-ray powder diffraction (XRD) pattern of the nitrogen-containing carbon dot comprises characteristic peaks at 2θ values of 19.12°±0.2°, 19.44°±0.2°, 19.8°±0.2°, 20.04°±0.2°, and/or 20.28°±0.2°.

In at least one embodiment of the present disclosure, an X-ray powder diffraction (XRD) pattern of the nitrogen-containing carbon dot comprises a broad peak at a 2θ value of 42°±0.2°.

In at least one embodiment of the present disclosure, the zeta potential of the nitrogen-containing carbon dot may range from about −1 mV to about 20 mV. Non-limiting examples include about −0.9 m V to about 19 mV, about −0.8 m V to about 18 mV, or about −0.7 mV to about 17 mV. In some embodiments, the zeta potential may be about −1 mV, −0.9 mV, −0.8 mV, −0.7 mV, −0.6 mV, −0.5 mV, −0.4 mV, −0.3 mV, −0.2 mV, −0.1 mV, 0 mV, 0.1 mV, 0.2 mV, 0.3 mV, 0.4 mV, 0.5 mV, 0.6 mV, 0.7 mV, 0.8 mV, 0.9 mV, 1 mV, 1.2 mV, 1.4 mV, 1.6 mV, 1.8 mV, 2 mV, 2.2 mV, 2.4 mV, 2.6 mV, 2.8 mV, 3 mV, 3.2 mV, 3.4 mV, 3.6 mV, 3.8 mV, 4 mV, 4.5 mV, 5 mV, 6 mV, 7 mV, 8 mV, 9 mV, 10 mV, 11 mV, 12 mV, 13 mV, 14 mV, 15 mV, 16 mV, 17 mV, 18 mV, 19 mV, or 20 mV. However, the present disclosure is not limited thereto.

In at least one embodiment of the present disclosure, the nitrogen-containing carbon dot may be a dendritic carbon dot. In some embodiments, the dendritic carbon dot may have a non-conjugated structure.

In at least one embodiment of the present disclosure, the nitrogen-containing carbon dot may be formed from an A2-type monomer and a B3-type monomer. In some embodiments, the A2-type monomer may be a dianhydride, and the B3-type monomer may be a polyetheramine. In some embodiments, the nitrogen-containing carbon dot may be formed by subjecting the A2-type monomer and the B3-type monomer to a one-pot synthesis process. In some embodiments, the nitrogen-containing carbon dot may be formed by subjecting the A2-type monomer and the B3-type monomer to a one-pot synthesis process and hydrothermal heating.

In at least one embodiment of the present disclosure, the nitrogen content in the nitrogen-containing carbon dot may range from about 5 atomic percentage (at %) to about 10 at %. Non-limiting examples include about 5.2 at % to about 9.8 at %, about 5.4 at % to about 9.6 at %, or about 5.6 at % to about 9.4 at %. In some embodiments, the nitrogen content may be about 5 at %, 5.2 at %, 5.4 at %, 5.6 at %, 5.8 at %, 6 at %, 6.2 at %, 6.4 at %, 6.6 at %, 6.8 at %, 7 at %, 7.2 at %, 7.4 at %, 7.6 at %, 7.8 at %, 8 at %, 8.2 at %, 8.4 at %, 8.6 at %, 8.8 at %, 9 at %, 9.2 at %, 9.4 at %, 9.6 at %, 9.8 at %, or 10 at %. However, the present disclosure is not limited thereto.

In at least one embodiment of the present disclosure, the particle size of the nitrogen-containing carbon dot may be greater than about 10 nm. In some embodiments, the particle size of the carbon dot may be greater than about 10 nm, 15 nm, 20 nm, 25 nm, 30 nm, 35 nm, 40 nm, 45 nm, 50 nm, 75 nm, 100 nm, 125 nm, 150 nm, 175 nm, 200 nm, 225 nm, 250 nm, 275 nm, 300 nm, 325 nm, 350 nm, 375 nm, 400 nm, 425 nm, 450 nm, 475 nm, 500 nm, 525 nm, 550 nm, 575 nm, or 600 nm. However, the present disclosure is not limited thereto. In some embodiments, the particle size of the nitrogen-containing carbon dot may range from about 10 nm to about 600 nm, about 20 nm to about 550 nm, or about 30 nm to about 500 nm. However, the present disclosure is not limited thereto.

In at least one embodiment of the present disclosure, the crystal plane of the nitrogen-containing carbon dot corresponding to the graphite (002) plane may have an interplanar spacing of 4 Å to about 6 Å. Non-limiting examples include about 4.1 Å to about 5.9 Å, about 4.2 Å to about 5.8 Å, or about 4.3 Å to about 5.7 Å. In some embodiments, the crystal plane of the nitrogen-containing carbon dot corresponding to the graphite (002) plane may have an interplanar spacing of about 4.1 Å, 4.2 Å, 4.3 Å, 4.4 Å, 4.5 Å, 4.6 Å, 4.7 Å, 4.8 Å, 4.9 Å, 5 Å, 5.1 Å, 5.2 Å, 5.3 Å, 5.4 Å, 5.5 Å, 5.6 Å, 5.7 Å, 5.8 Å, 5.9 Å, or 6 Å. However, the present disclosure is not limited thereto.

In at least one embodiment of the present disclosure, the crystal plane of the nitrogen-containing carbon dot corresponding to the graphite (100) plane may have an interplanar spacing of about 1 Å to about 3 Å. Non-limiting examples include about 1.1 Å to about 2.9 Å, about 1.2 Å to about 2.8 Å, or about 1.3 Å to about 2.7 Å. In some embodiments, the crystal plane of the nitrogen-containing carbon dot corresponding to the graphite (100) plane may have an interplanar spacing of about 1.1 Å, 1.2 Å, 1.3 Å, 1.4 Å, 1.5 Å, 1.6 Å, 1.7 Å, 1.8 Å, 1.9 Å, 2 Å, 2.1 Å, 2.2 Å, 2.3 Å, 2.4 Å, 2.5 Å, 2.6 Å, 2.7 Å, 2.8 Å, 2.9 Å, or 3 Å. However, the present disclosure is not limited thereto.

In at least one embodiment of the present disclosure, after subjecting the A2-type monomer and the B3-type monomer to the one-pot synthesis, the A2-type monomer and B3-type monomer may be heated by a hydrothermal method.

In at least one embodiment of the present disclosure, the treatment of the A2-type monomer and the B3-type monomer by the one-pot synthesis may include: dissolving the A2-type monomer to form a first solution; dissolving the B3-type monomer to form a second solution; mixing the first solution and the second solution to form a mixed solution comprising a precipitate; and collecting and drying the precipitate.

In at least one embodiment of the present disclosure, heating the treated A2-type monomer and B3-type monomer by the hydrothermal method may include: diluting the precipitate to form a diluted solution; heating the diluted solution; collecting and filtering the supernatant of the diluted solution; and drying the supernatant.

In at least one embodiment of the present disclosure, the A2-type monomer is a dianhydride. In some embodiments, the A2-type monomer may be a dianhydride having a bridged bicyclic alkene, such as, but not limited to: bicyclo[2.2.2]oct-7-ene-2,3,5,6-tetracarboxylic dianhydride (BCDA); ethylenetetracarboxylic dianhydride (ETDA); or a dianhydride having a non-alkene, such as, but not limited to: ethylenediaminetetraacetic dianhydride (EDTAD); cyclobutane-1,2,3,4-tetracarboxylic acid dianhydride (CBDA); 1,2,4,5-cyclohexanetetracarboxylic dianhydride (CHDA); 1,2,3,4-cyclopentanetetracarboxylic dianhydride (CPDA); dicyclohexyl-3,4,3′,4′-tetracarboxylic dianhydride (DHTDA); diethylenetriaminepentaacetic acid dianhydride (DEPDA); or meso-butane-1,2,3,4-tetracarboxylic dianhydride (BTDA).

In at least one embodiment of the present disclosure, the B3-type monomer is a polyetheramine, such as, but not limited to: T-403 (i.e., Jeffamine® T-403), T-3000 (i.e., Jeffamine® T-3000), or T-5000 (i.e., Jeffamine® T-5000).

In at least one embodiment of the present disclosure, heating the diluted solution may include: raising the temperature of the diluted solution to the temperature of about 150° C. to about 250° C. under a pressure of about 100 psi to about 120 psi; and maintaining the heating temperature for about 1 hour to about 10 hours.

In at least one embodiment of the present disclosure, the pressure may be about 100 psi to about 120 psi, such as, but not limited to: about 101 psi to about 117 psi, about 102 psi to about 118 psi, or about 103 psi to about 119 psi. In some embodiments, the pressure may be about 100 psi, 101 psi, 102 psi, 103 psi, 104 psi, 105 psi, 106 psi, 107 psi, 108 psi, 109 psi, 110 psi, 111 psi, 112 psi, 113 psi, 114 psi, 115 psi, 116 psi, 117 psi, 118 psi, 119 psi, or 120 psi, but the present disclosure is not limited thereto.

In at least one embodiment of the present disclosure, the heating temperature may be about 150° C. to about 250° C., such as, but not limited to: about 155° C. to about 245° C., about 160° C. to about 240° C., or about 165° C. to about 235° C. In some embodiments, the heating temperature may be about 150° C., 155° C., 160° C., 165° C., 170° C., 175° C., 180° C., 185° C., 190° C., 195° C., 200° C., 205° C., 210° C., 215° C., 220° C., 225° C., 230° C., 235° C., 240° C., 245° C., or 250° C., but the present disclosure is not limited thereto.

In at least one embodiment of the present disclosure, the heating time may be about 1 hour to about 10 hours, such as, but not limited to: about 1.5 hours to about 9.5 hours, about 2 hours to about 9 hours, or about 2.5 hours to about 8.5 hours. In some embodiments, the heating time may be about 1 hour, 1.5 hours, 2 hours, 2.5 hours, 3 hours, 3.5 hours, 4 hours, 4.5 hours, 5 hours, 5.5 hours, 6 hours, 6.5 hours, 7 hours, 7.5 hours, 8 hours, 8.5 hours, 9 hours, 9.5 hours, or 10 hours, but the present disclosure is not limited thereto.

In at least one embodiment of the present disclosure, the yield rate of the polymer dots of the present disclosure may range from about 5% to about 95%, about 20% to about 90%, about 30% to about 80%, or about 40% to about 70%, but the present disclosure is not limited thereto. In some embodiments, the yield rate of the polymer dots of the present disclosure may be about 5%, 10%, 15%, 20%, 25%, 30%, 35%, 40%, 45%, 50%, 55%, 60%, 65%, 70%, 75%, 80%, 85%, 90%, or 95%, but the present disclosure is not limited thereto.

In at least one embodiment of the present disclosure, the recovery rate of the polymer dots of the present disclosure may range from about 70% to about 99%, about 75% to about 98%, about 80% to about 97%, or about 85% to about 96%, but the present disclosure is not limited thereto. In some embodiments, the recovery rate of the polymer dots of the present disclosure may be about 70%, 75%, 80%, 85%, 86%, 87%, 88%, 89%, 90%, 91%, 92%, 93%, 94%, 95%, 96%, 97%, 98%, or 99%, but the present disclosure is not limited thereto.

In at least one embodiment of the present disclosure, a method for suppressing at least one of inflammation, bacterial growth, or oxidative stress is provided. The method comprises administering an effective amount of the polymer dots to a subject in need thereof.

In at least one embodiment of the present disclosure, the polymer dots are used in the preparation of a composition for at least one of anti-inflammatory, antibacterial, or antioxidant purposes. The composition may further comprise a pharmaceutically or cosmetically acceptable carrier. In some embodiments, the composition may be in the form of a solid, liquid, colloid, or aerosol, such as, but not limited to: dry powder, tablet, granule, capsule, film, injectable formulation, oral solution, gel, ointment, paste, or aerosol spray, but the present disclosure is not limited thereto.

In at least one embodiment of the present disclosure, the polymer dots with different degrees of crosslinking are provided. The carbon core of the polymer dots exhibits different bandgaps due to variations in carbon arrangement. The changes in the bandgap and the fluorescence induced by crosslinked clusters facilitate the formation of tunable fluorescent polymer dots. The polymer dots exhibit advantages such as low cost, ease of preparation, excellent photostability, excellent biocompatibility, and nanoscale size.

In at least one embodiment of the present disclosure, the polymer dots exhibit intrinsic fluorescence and antioxidant activity, enabling their use as diagnostic probes or therapeutic agents in the field of bioimaging. In some embodiments, the polymer dots further exhibit antibacterial activity and, through their antibacterial and/or antioxidant effects, may be used to treat oxidative stress-related diseases, such as, but not limited to, inflammatory skin disorders.

In at least one embodiment of the present disclosure, the polymer dots of the present disclosure overcome the clinical application limitations associated with conventional antioxidant materials, such as natural enzymes derived from natural sources, including high production costs, low yields, structural instability, compositional complexity, and large molecular size that restricts cellular permeability.

### EXAMPLES

The following examples are provided to further illustrate specific embodiments of the present disclosure and are not intended to limit the scope of the present disclosure in any way.

### Preparation Example

Under a nitrogen atmosphere, ethylenediaminetetraacetic dianhydride (EDTAD) (0.5 g, 2 mmol) was dissolved in 100 mL of N,N-dimethylformamide (DMF) to form an EDTAD solution, and Jeffamine® T-403 (1.8 g, 4 mmol) was dissolved in 100 mL of tetrahydrofuran (THF) under a nitrogen atmosphere to form a T-403 solution. The EDTAD solution was then slowly added dropwise into the T-403 solution in an ice bath at 0° C. to form a mixed solution. The mixed solution was gradually warmed to room temperature and allowed to stand for condensation polymerization. The total reaction time was 20 hours. Upon completion of the reaction, the resulting precipitate was collected and dried under vacuum at 50° C. until a constant weight was achieved to obtain the polymer dots of the preparation example.

### Example 1

The polymer dots of the preparation example were diluted to form a 25 mg/mL of diluted solution. The diluted solution was transferred into a high-temperature, high-pressure reactor, and nitrogen gas was subsequently introduced into the reactor. The reactor was then placed in a glycerol bath and heated to a temperature of 180° C. under a pressure of 110 psi for 3 hours to carry out the heating reaction. After the reaction was completed, the solution was centrifuged at 6000 rpm for 30 minutes to collect the supernatant, which was then filtered through a 0.22 μm membrane. The filtered supernatant was concentrated under vacuum at 75° C. using a rotary evaporator to obtain a viscous substance, which was subsequently dried under vacuum at 50° C. in a vacuum oven until a constant weight was achieved to obtain the polymer dots of Example 1.

### Example 2

The polymer dots of Example 2 were prepared using the method described in Example 1, except that the heating time was modified to 6 hours.

### Example 3

The polymer dots of Example 3 were prepared using the method described in Example 1, except that the heating temperature was modified to 220° C.

### Example 4

The polymer dots of Example 4 were prepared using the method described in Example 3, except that the heating time was modified to 6 hours.

It should be noted that the polymer dots of the above Preparation Example and Examples 1 to 4 all possess a wholly aliphatic structure, and the only difference lies in the degree of crosslinking.

### Analysis and Results

### 1. Fluorescence Performance

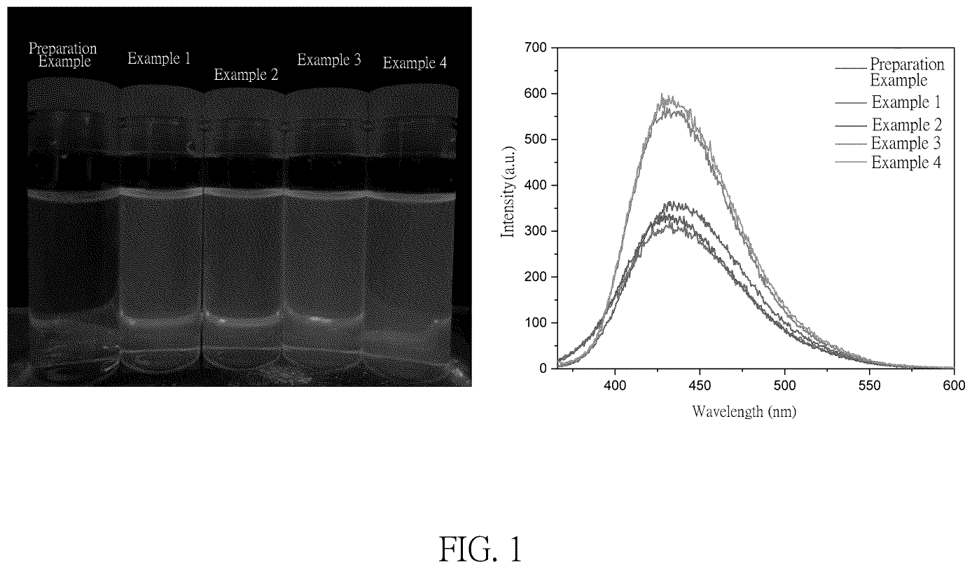

Aqueous solutions of the polymer dots from the Preparation Example and Examples 1 to 4 were diluted to 0.25 mg/mL. The spontaneous fluorescence capability of the polymer dots was observed by irradiating the solutions with a UV lamp at a wavelength of 365 nm. The results are shown in the left part of FIG. 1.

Additionally, aqueous solutions of the polymer dots from the Preparation Example and Examples 1 to 4 were diluted to 2 mg/mL. A fluorescence spectrometer (LS-50B, PerkinElmer) was used to measure the photoluminescence (PL) spectra of the solutions to evaluate the fluorescence intensity of the polymer dots upon excitation at a wavelength of 360 nm. The results are shown in the right part of FIG. 1.

Referring to the left part of FIG. 1, the aqueous solutions of the polymer dots from the Preparation Example and Examples 1 to 4 exhibited blue fluorescence, with those from Examples 1 to 4 showing particularly prominent blue fluorescence. The right part of FIG. 1 demonstrates that all samples exhibited fluorescence intensity, with Examples 3 and 4 displaying higher fluorescence intensity values. These results confirm that the polymer dots of the present disclosure possess excellent spontaneous fluorescence performance due to the crosslink-enhanced emission (CEE) effect.

### 2. Morphology, Particle Size, and Zeta Potential

### 2.1 Transmission Electron Microscopy (TEM) Analysis

Aqueous solutions of the polymer dots from the Preparation Example and Examples 1 to 4 were diluted to 10 mg/mL. A carbon-coated copper grid was immersed in the aqueous solution with gentle back-and-forth motion for 1 to 2 minutes, then removed and blotted dry around the edges with lens paper to remove excess solution. The grid was left to dry in a desiccator overnight and subjected to transmission electron microscopy (TEM) imaging analysis (JEM-1400 FLASH, JEOL) to observe the morphology and particle size of the polymer dots. The measured particle sizes are recorded in Table 1.

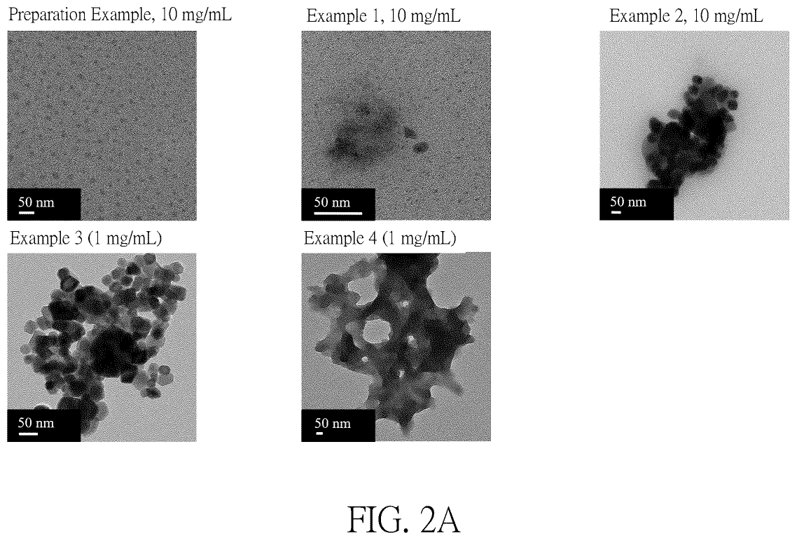

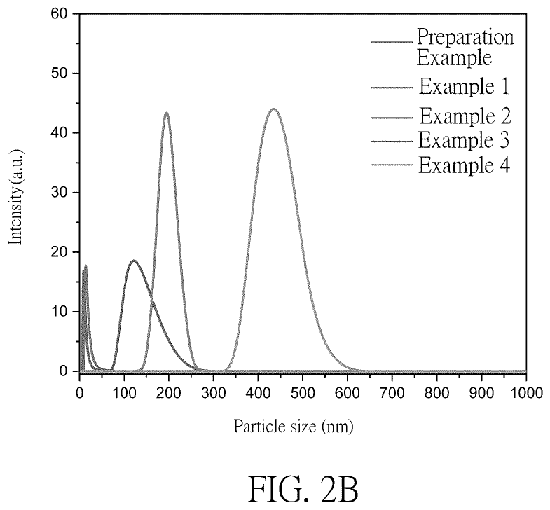

As shown in the upper left part of FIG. 2A, the polymer dots from the Preparation Example exhibit uniform spherical morphology. As shown in the upper middle, upper right, and lower left parts of FIG. 2A, the polymer dots from Examples 1, 2, and 3 aggregate into clusters due to mild crosslinking. As shown in the lower middle part of FIG. 2A, the polymer dots from Example 4 form sheet-like aggregates due to more extensive crosslinking.

### 2.2 Dynamic Light Scattering (DLS) Analysis

Aqueous solutions of the polymer dots from the Preparation Example and Examples 1 to 4 were diluted to 1 mg/mL and placed into plastic cuvettes for analysis using a dynamic light scattering particle size analyzer (Zetasizer Nano ZS90, Malvern). The particle size and zeta potential of the polymer dots were analyzed to determine the average particle size distribution and zeta potential. The results are shown in FIG. 2B, and the particle size, average size distribution, and zeta potential are summarized in Table 1.

TABLE 1

| | Particle Size of | Average Particle Size | Zeta potential |
|---|---|---|---|
| | Polymer Dot (nm) | Distribution (nm) | (mV) |
| Preparation | 5 to 10 | 8.8 ± 2.1 | −0.6 ± 0.1 |
| Example | | | |
| Example 1 | 10 to 15 | 16.1 ± 1 | 0.1 ± 0.1 |
| Example 2 | 40 to 50 | 133.5 ± 18.2 | 1.2 ± 0.2 |
| Example 3 | 25 to 100 | 163.4 ± 36.6 | 15.5 ± 0.3 |
| Example 4 | >200 | 420.3 ± 18.1 | 3.4 ± 0.2 |

As shown in Table 1, the particle size of the polymer dots of the present disclosure increases with increasing heating temperature and heating duration.

### 3. Main Elemental Component, Content, and Bond Strength

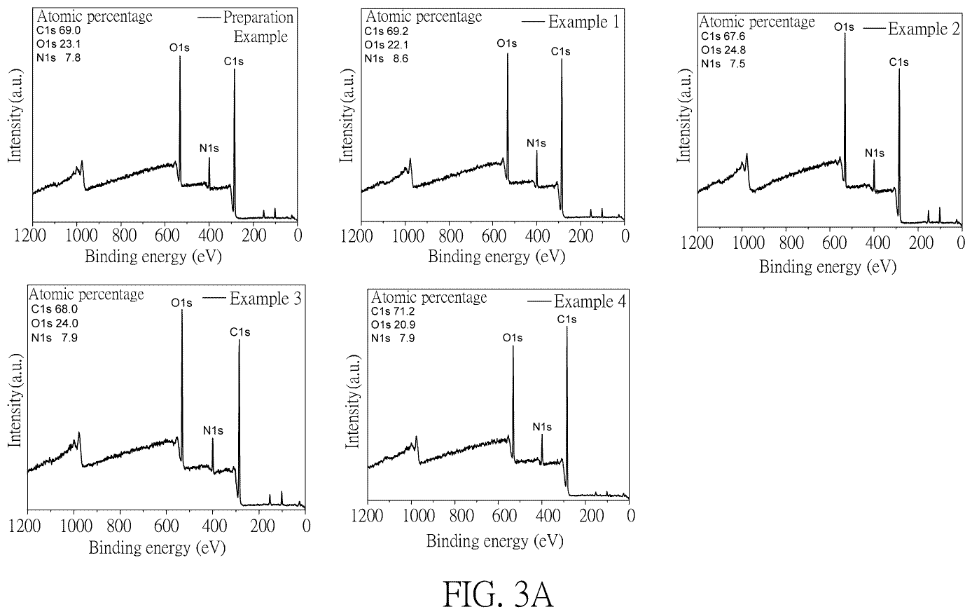

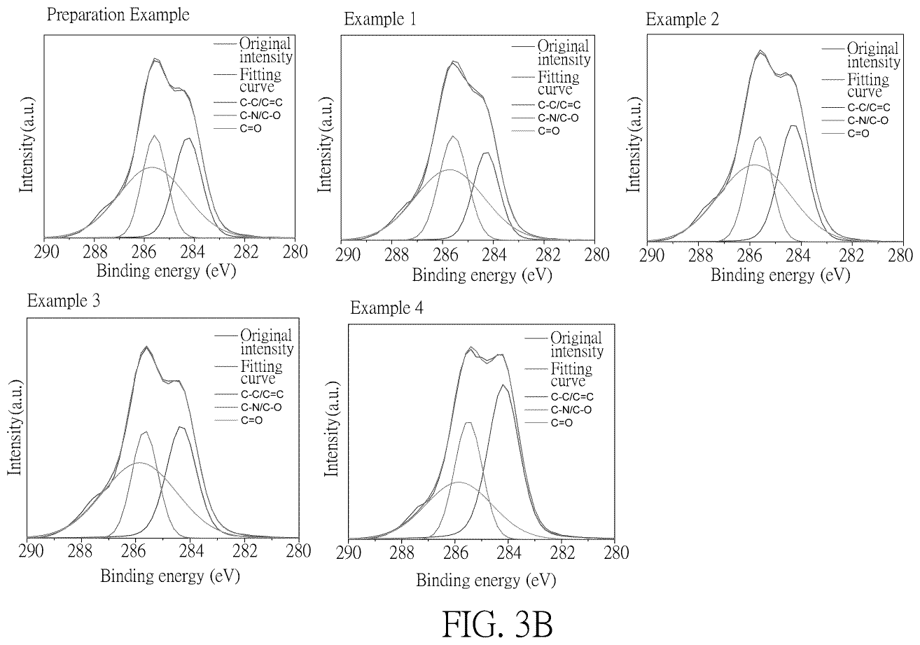

One drop of deionized water was added to the polymer dot samples from the Preparation Example and Examples 1 to 4 to form a viscous material. Microscope slides were cut to 0.5 cm×0.5 cm, and the viscous sample was uniformly applied to the slide with a coating thickness of approximately 0.3 cm. After coating, the samples were placed in a vacuum oven and dried at 50° C. under vacuum until the day before submission for x-ray photoelectron spectroscopy (XPS) analysis. The results are shown in FIG. 3A and FIG. 3B, and the elemental component and content are summarized in Table 2.

TABLE 2

| Main Elemental | | | |
|---|---|---|---|
| Component Content | Carbon (%) | Oxygen (%) | Nitrogen (%) |
| Preparation Example | 69.0 | 23.1 | 7.8 |
| Example 1 | 69.2 | 22.1 | 8.6 |
| Example 2 | 67.6 | 24.8 | 7.5 |
| Example 3 | 68.0 | 24.0 | 7.9 |
| Example 4 | 71.2 | 20.9 | 7.9 |

Referring to FIG. 3A and Table 2, the main elemental components of the polymer dots of the Preparation Example and Examples 1 to 4 are carbon (C), oxygen (O), and nitrogen (N), and the content ratios are similar.

Referring to the C1s spectra in FIG. 3B, the polymer dots of the Preparation Example and Examples 1 to 4 exhibit a C—C/C═C signal at approximately 283.8 eV, a C—N/C—O signal at approximately 285.1 eV, and a C═O signal at approximately 286.1 eV. Among them, due to bonding changes caused by heating, the C—C/C═C signal of the polymer dots in Example 1 is slightly reduced because the bonding has not yet stabilized. In contrast, the polymer dots in Examples 2 to 4 exhibit stronger C—C/C═C signals, indicating the formation of stronger covalent bonds between carbon atoms and a tighter molecular structure, thereby confirming an increased degree of crosslinking of the polymer dots.

### 4. Crystal Structure

### 4.1 X-ray Diffraction (XRD) Analysis

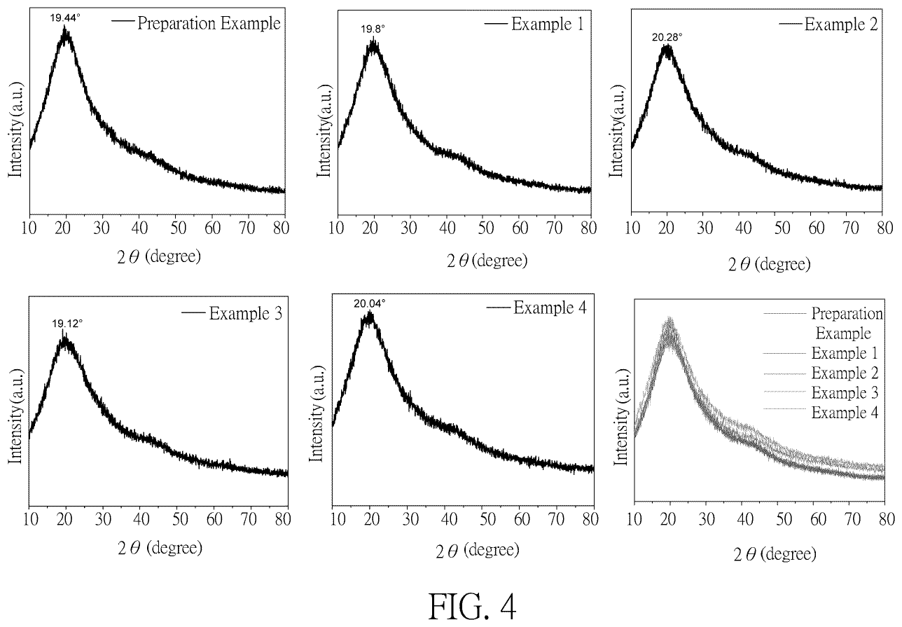

One drop of deionized water was added to each polymer dot sample of the Preparation Example and Examples 1 to 4 to render it into a viscous state. The viscous sample was evenly spread onto a glass slide using a micropipette to form a coating area of 1 cm×1 cm and a thickness less than 5 mm. After coating, the sample was vacuum-dried in an oven at 50° C. until one day before analysis. The crystalline structure was then identified using an x-ray diffractometer (XRD), and the results are shown in FIG. 4. The diffraction peak positions are recorded in Table 3 below.

### 4.2 Bragg's Law

Based on Bragg's Law as expressed in Equation (I) below, the interplanar spacings d(002) and d(100) of the polymer dots in the Preparation Example and Examples 1 to 4 were calculated. These correspond to the (002) and (100) planes of graphite, respectively, and are summarized in Table 3 below.

$$n λ = 2 d sin ( θ ) \qquad ( Equation I )$$

$$\begin{matrix}n : Postitive integer \\ λ : Wavelength \\ d : Interplanar spacing \\ θ : Incidence angle\end{matrix}$$

TABLE 3

| | | | Broadened | |
|---|---|---|---|---|
| | Diffraction | | Diffraction | |
| | Peak (°) | d₍₀₀₂₎(Å) | Peak (°) | d₍₁₀₀₎(Å) |
| Preparation | 19.44 ± 0.2 | 4.6 | — | — |
| Example | | | | |
| Example 1 | 19.8 ± 0.2 | 4.5 | 42 ± 0.2 | 2.2 |
| Example 2 | 20.28 ± 0.2 | 4.4 | 42 ± 0.2 | 2.2 |
| Example 3 | 19.12 ± 0.2 | 4.6 | 42 ± 0.2 | 2.2 |
| Example 4 | 20.04 ± 0.2 | 4.4 | 42 ± 0.2 | 2.2 |

Referring to FIG. 4 and Table 3, the polymer dots of the Preparation Example and Examples 1 to 4 exhibit a relatively strong characteristic peaks at 20 values of about 18° to about 21°, indicating that the crystal of the polymer dots possess a crystal plane corresponding to the graphite (002) plane.

The interplanar spacing of the conventional graphite (002) plane is about 3.5 Å, whereas the interplanar spacing d(002) of the polymer dots of the Preparation Example and Examples 1 to 4 is about 4 Å to 6 Å. Compared to the graphite (002) plane, the polymer dots exhibit a larger d(002) interplanar spacing range. These results confirm that the polymer dots have functional groups on their surfaces or edges, and the steric hindrance of these functional groups contributes to the increased interplanar spacing.

FIG. 4 and Table 3 further show that the polymer dots of Examples 1 to 4 exhibit a relatively weak and broadened peak at a 20 value of about 42°, indicating that the crystals of the polymer dots possess a crystal plane corresponding to the graphite (100) plane, with an interplanar spacing of about 2.2 Å.

### 5. Cytotoxicity

### 5.1 Cytotoxicity Analysis of Polymer Dots on Murine Macrophage Cells (RAW 264.7 Cells)

The polymer dots of the Preparation Example and Examples 1 to 4 were diluted to prepare aqueous solutions with concentrations of 0 μg/mL, 125 μg/mL, 250 μg/mL, 500 μg/mL, and 1000 μg/mL.

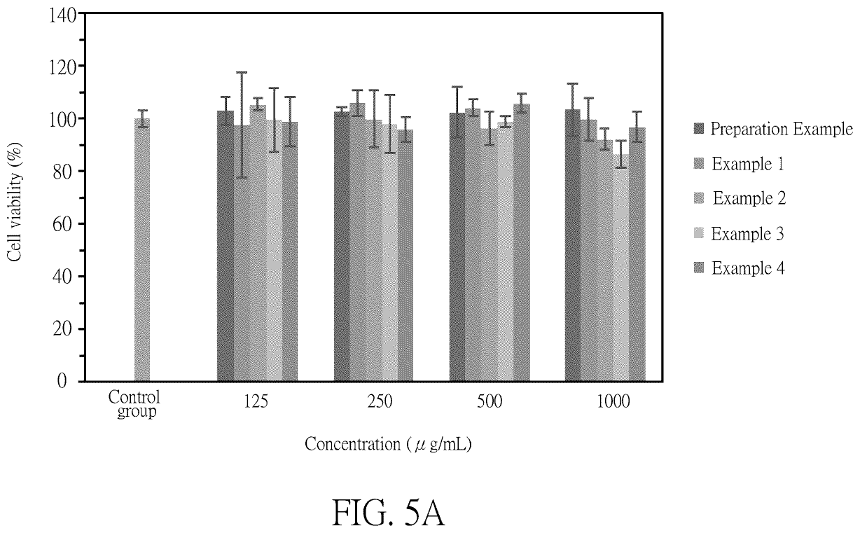

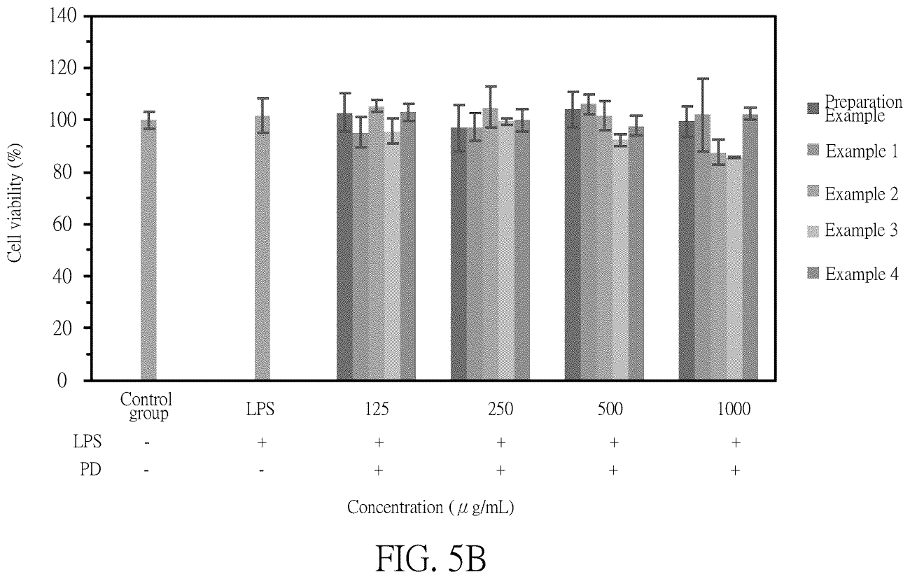

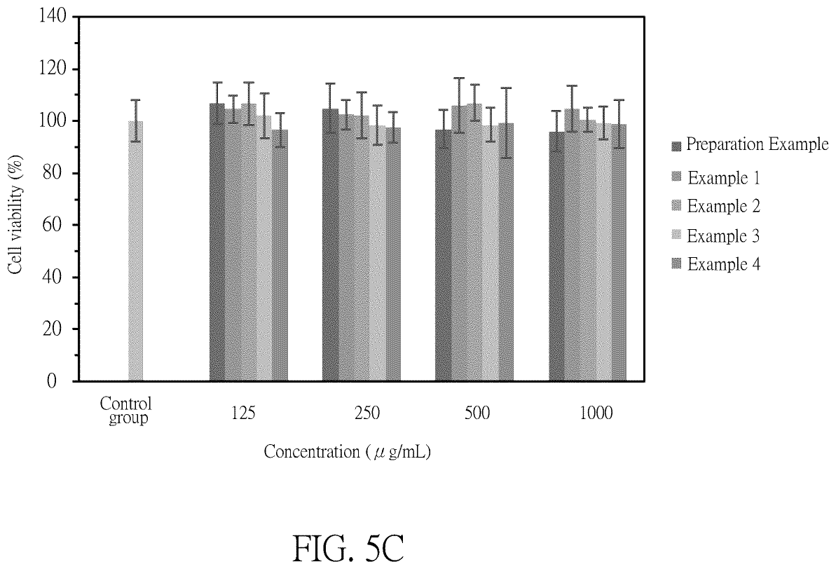

RAW 264.7 cells, as well as RAW 264.7 cells induced in vitro with LPS to induce an inflammatory response producing nitric oxide (NO), were seeded at 5×10⁴ cells per well in a 96-well plate and cultured for 24 hours. The cells were treated with the above aqueous solutions for 24 hours to form mixtures. The culture medium was then replaced with a fresh medium containing 0.5 mg/mL of 3-(4,5-dimethylthiazol-2-yl)-2,5-diphenyltetrazolium bromide (MTT) (purchased from Bio Basic), followed by incubation at 37° C. for 4 hours. After the MTT reaction, the supernatant was removed, and 200 μL of dimethyl sulfoxide (DMSO) (purchased from Honeywell International Inc.) was added. The absorbance was measured at OD570 using a SpectraMax® M2e microplate reader. The results are shown in FIGS. 5A and 5B.

Referring to FIG. 5A, the polymer dots of the Preparation Example and Examples 1 to 4 maintained cell viability above 80% at concentrations as high as 1000 μg/mL in RAW 264.7 cells. These results confirm that the polymer dots of the present disclosure exhibit low cytotoxicity and good biocompatibility.

Referring to FIG. 5B, the polymer dots of the Preparation Example and Examples 1 to 4 maintained RAW 264.7 cell viability above 80% at concentrations up to 1000 μg/mL, even under LPS-induced inflammatory conditions that promote nitric oxide (NO) production. This confirms the polymer dots exhibit low cytotoxicity and good biocompatibility. In particular, the polymer dots of Example 1 demonstrated the most significant improvement in cell viability at 500 μg/mL, indicating a superior effect in reducing inflammation-related markers induced by LPS and maintaining normal cellular metabolism.

### 5.2 Cytotoxicity Analysis of Polymer Dots on Human Hepatocellular Carcinoma Cells (HepG2)

The polymer dots of the Preparation Example and Examples 1 to 4 were diluted to prepare aqueous samples at concentrations of 0 μg/mL, 125 μg/mL, 250 μg/mL, 500 μg/mL, and 1000 μg/mL.

HepG2 cells were seeded at 5,000 cells per well in a 96-well plate and cultured in fresh Dulbecco's Modified Eagle Medium (DMEM) containing 10% fetal bovine serum (FBS) and 1% antibiotics at 37° C. and 5.0% CO₂.

Each aqueous sample was mixed with phosphate buffered saline (PBS) to a final concentration of 20 mg/mL, filtered through a 0.22 μm membrane, and diluted into DMEM to form polymer dot-containing solutions of various concentrations. The HepG2 cells were treated with these solutions for 48 hours. Afterward, 20 μL of 5 mg/mL MTT stock solution was added to each well and reacted for 2-4 hours. The supernatant was removed, and 200 μL of dimethyl sulfoxide (DMSO) was added. Absorbance was measured at OD₅₇₀ using the SpectraMaxx M2e microplate reader. The results are shown in FIG. 5C.

As shown in FIG. 5C, the polymer dots of the Preparation Example and Examples 1 to 4 maintained HepG2 cell viability above 80% even at a concentration of 1000 μg/mL. These results confirm that the polymer dots exhibit low cytotoxicity, good biocompatibility, and no acute hepatotoxicity.

### 6. Analysis of Nitric Oxide Scavenging Capacity

The polymer dots of the Preparation Example and Examples 1 to 4 were diluted to prepare aqueous samples at concentrations of 0 μg/mL, 125 μg/mL, 250 μg/mL, 500 μg/mL, and 1000 μg/mL.

RAW 264.7 cells were cultured in DMEM supplemented with 10% FBS, 100 U/mL penicillin and streptomycin, and 4 mM L-glutamine in a humidified incubator at 37° C. with 5% CO₂.

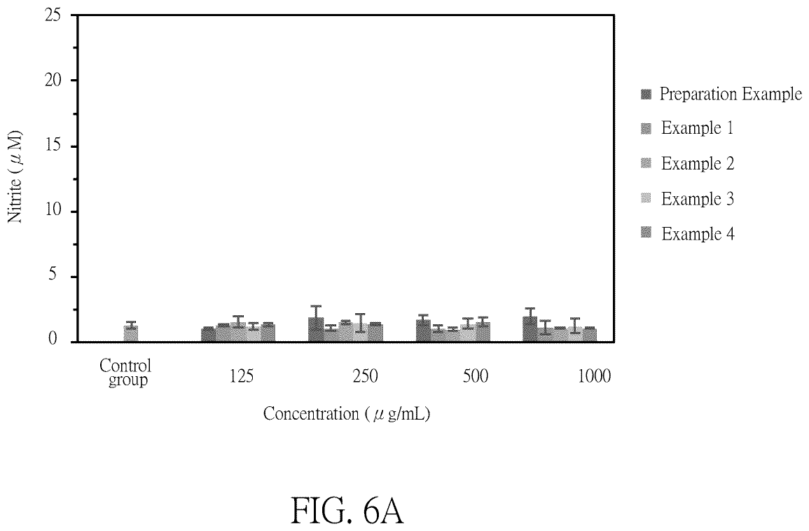

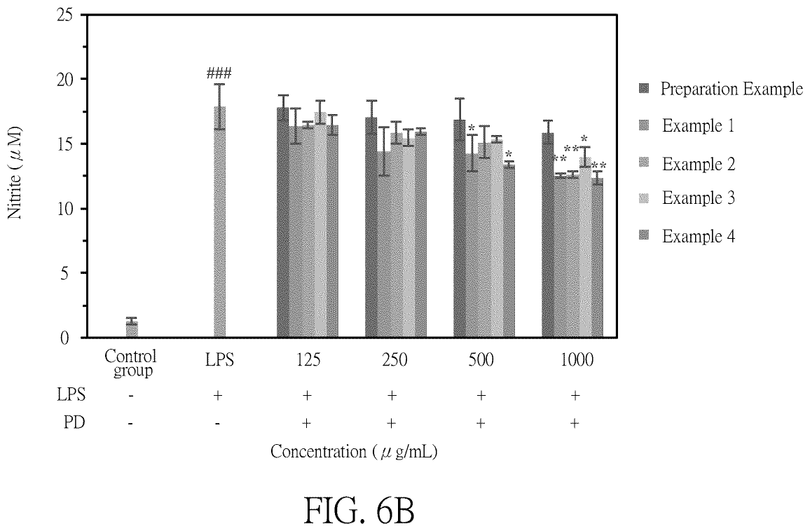

RAW 264.7 cells were seeded at 5×10⁴ cells per well in a 96-well plate and cultured for 24 hours. The cells were treated with the aqueous polymer dot samples for 24 hours. Lipopolysaccharide (LPS) (100 ng/mL) was used as a positive control to induce inflammation and nitric oxide production. The supernatants (i.e., the solutions removed following the MTT assay) were collected and mixed with an equal volume of Griess reagent (1% sulphanilamide, 0.1% naphthylethylenediamine dihydrochloride, and 5% phosphoric acid). The absorbance was measured at OD570 using the SpectraMax® M2e to evaluate nitric oxide scavenging ability. The results are shown in FIG. 6A.

Referring to FIG. 6A, the nitric oxide scavenging capacity of the polymer dots from the Preparation Example and Examples 1 to 4 did not show significant differences compared with the control group, indicating that the polymer dots of the present disclosure do not induce inflammatory responses.

Similarly, the present disclosure further assessed the nitric oxide scavenging ability under a simulated inflammatory condition by using LPS to induce inflammation. The results are shown in FIG. 6B.

Referring to FIG. 6B, the nitric oxide scavenging ability of the polymer dots of the Preparation Example and Examples 1 to 4 increased with elevated heating temperature and duration. While a moderate level of nitric oxide plays beneficial roles in wound healing, such as pathogen elimination, angiogenesis, and promotion of epidermal and endothelial cell proliferation, excessive nitric oxide can induce oxidative stress, damage cells or tissues, and lead to inflammation and necrosis. Therefore, the excellent nitric oxide scavenging capability of the polymer dots of the present disclosure confirms their anti-inflammatory potential.

### 7. Antibacterial Activity

The antibacterial effect of the polymer dots against Escherichia coli (E. coli) was evaluated using the disk diffusion method.

First, the polymer dots of the Preparation Example, Example 1, and Example 2 were diluted to prepare aqueous samples at a concentration of 50 mg/mL.

E. coli was streaked onto LB agar medium and incubated at 37° C. for 16 to 18 hours. A single colony was then inoculated into 3 mL of LB broth and pre-activated by shaking at 250 rpm and 37° C. for 16 to 18 hours. Then, 10 μL of pre-activated bacterial culture was inoculated into 3 mL of LB broth and cultured for 2 to 3 hours under the same conditions. The optical density (OD₆₀₀) was measured, and when an appropriate value was reached, 1.3 mL of the culture was transferred to a microcentrifuge tube. The culture was centrifuged at 10,000 rpm for 10 to 15 minutes at room temperature, the supernatant was discarded, and the bacterial pellet was resuspended in 1 mL of sterile water to yield a bacterial suspension with a final concentration of approximately 1.00×10⁷ to 1.00×10⁸ CFU/mL. Then, 100 μL of this suspension was spread evenly over the LB agar plate until no liquid pooled on the surface. Filter paper disks were placed on the agar surface. To each disk, 40 L of sterile water (negative control), 0.25 mg/mL ampicillin (positive control), or polymer dot sample was added. The plates were incubated at 37° C. for 24 hours under mild shaking, and inhibition zones were observed.

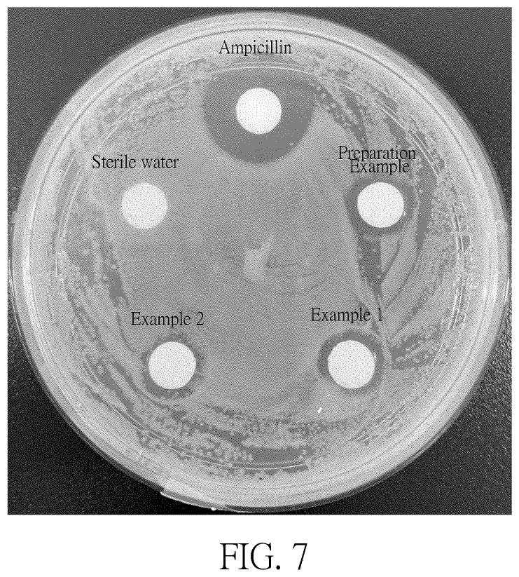

As shown in FIG. 7, the polymer dots of the Preparation Example, Example 1, and Example 2 all formed inhibition zones. This indicates that the polymer dots of the present disclosure can be absorbed by E. coli through simple diffusion, where they induce the generation of reactive oxygen species (ROS), resulting in oxidative stress, damage to the bacterial cell wall, and inhibition of bacterial growth and proliferation. Accordingly, the nitrogen-containing structure of the polymer dots confers bioactivity and antibacterial efficacy.

### 8. Free Radical Scavenging Ability

8.1 1-Diphenyl-2-picrylhydrazyl (DPPH) Free Radical

Aqueous solutions of the polymer dots from the Preparation Example and Examples 1 to 4 were prepared at concentrations ranging from 0 mg/mL to 40 mg/mL. A 50 μM ethanol solution of DPPH was prepared as the working solution, and water was used as the control group.

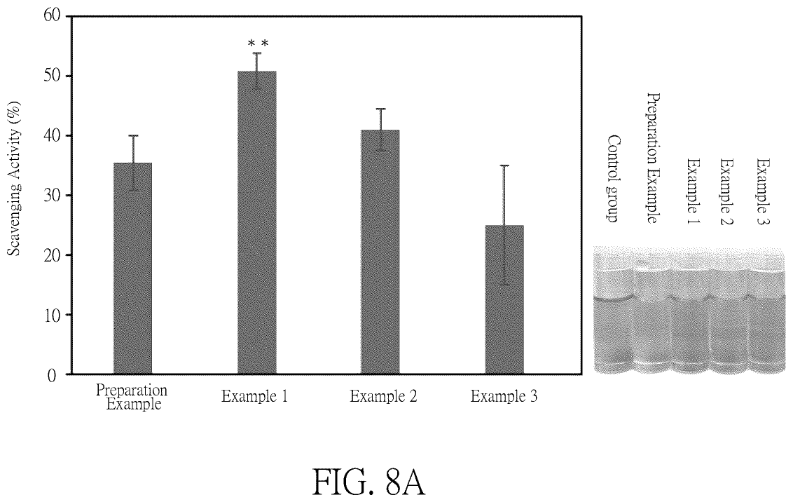

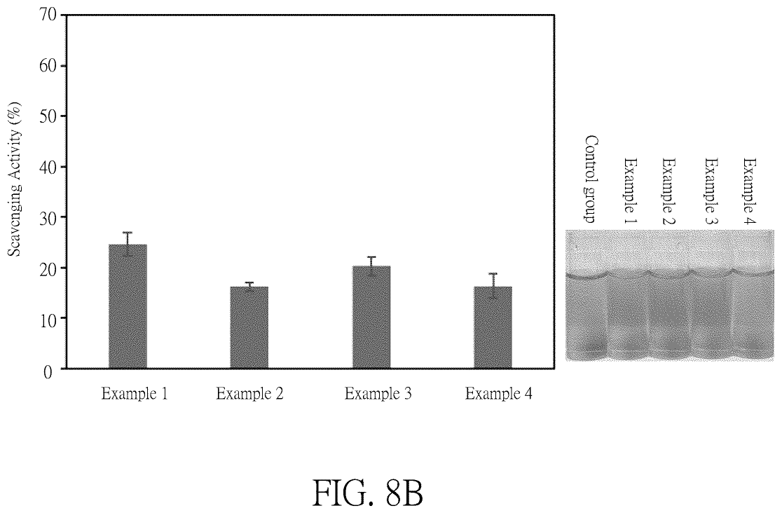

A 1 mL aliquot of the 50 μM DPPH ethanol solution was mixed uniformly with either water or each polymer dot sample of different concentrations, followed by the addition of 2 mL phosphate-buffered saline (PBS) to maintain a fixed final volume. The mixtures were reacted for 30 minutes in the dark. The absorbance at 517 nm was measured using a UV spectrophotometer. The DPPH radical scavenging activity was calculated using Equation (II), and the results are shown in FIG. 8A and Table 4 below.

$$D P P H radical scavenging activity ( \% ) = [ 1 - ( ( A t - A 1 ) / ( Ab - A 0 ) ) ] \times 100 \% \qquad ( Equation II )$$

$$Where :$$

$$\begin{matrix}At - A 1 : & Absorbance of sample at 517 nm \\ Ab - A 0 : & Absorbance of the control ( without sample ) at 517 nm\end{matrix}$$

TABLE 4

| | DPPH Radical Scavenging Activity (%) |
|---|---|
| | of Polymer Dots (1.75 mg/mL) |
| Preparation Example | 35.4 ± 4.6% |
| Example 1 | 50.7 ± 3.0% |
| Example 2 | 40.9 ± 3.5% |
| Example 3 | 24.9 ± 9.9% |

Referring to the left part of FIG. 8A and Table 4, the DPPH scavenging activity of the polymer dots of the present disclosure ranges from approximately 15% to approximately 60%, preferably from approximately 40% to approximately 60%. In addition, as shown in the right part of FIG. 8A, the control DPPH ethanol solution appears deep purple and exhibits maximum absorbance at a wavelength of 517 nm. The polymer dots of the Preparation Example and Examples 1 to 3 (Example 4 not shown) are capable of scavenging DPPH free radicals in the DPPH ethanol solution, resulting in a color change from deep purple to light yellow and a corresponding decrease in absorbance at 517 nm. This demonstrates that the polymer dots of the Preparation Example and Examples 1 to 4 possess antioxidant activity.

Based on the above results, the concentrations at which the polymer dots of the Preparation Example and Example 1 exhibit 50% scavenging activity, as well as their maximum scavenging activity, were further compared and are summarized in Table 5 below.

TABLE 5

| | A concentration with scavenging | Maximum scavenging |
|---|---|---|
| | activity up to 50% (mg/mL) | activity (%) |
| Preparation | 2.25 | 54.2 ± 6.9 |
| Example | | |
| Example 1 | 0.625 | 56.0 ± 4.3 |

As shown in Table 5, the polymer dots of Example 1 achieve a comparable scavenging rate to that of the Preparation Example at a lower concentration, indicating excellent DPPH radical scavenging capacity.

8.2 2,2′-Azino-bis(3-ethylbenzothiazoline-6-sulfonic acid) Diammonium Salt (ABTS) Cation (ABTS⋅+) Radical

The diluted polymer dots of the Preparation Example and Examples 1 to 4 were prepared as solutions ranging from 0.001 mg/mL to 0.2 mg/mL. An ABTS solution (14 mM) and a potassium persulfate solution (4.9 mM) were separately prepared. These solutions were mixed at a 1:1 volume ratio to form a mixture in which the final concentrations of ABTS and potassium persulfate were 7 mM and 2.45 mM, respectively. The mixture was allowed to react at 4° C. for 16 hours. The absorbance at 734 nm was then measured and adjusted to 0.7±0.02 L/(g cm). Water was used as the control.

1 mL of each sample (from the Preparation Example and Examples) was mixed with 1 mL of the ABTS mixture. Then, 2 mL of PBS buffer was added to fix the total volume. After reacting in the dark for 20 minutes, the absorbance at 734 nm was measured using a UV-visible spectrophotometer. The ABTS radical scavenging activity was calculated according to Equation (III) below. The results are shown in FIG. 8B and Table 6.

$$A B T S radical scavenging activity ( \% ) = [ ( A c - As ) / Ac ] \times 100 \% \qquad ( Equation III )$$

$$\begin{matrix}As : & Absorbance of the sample at 734 nm \\ Ac : & Absorbance of the blank control ( no sample ) at 734 nm\end{matrix}$$

TABLE 6

| | ABTS Radical Scavenging Activity (%) |
|---|---|
| Example 1 | 24.6 ± 2.4% |
| Example 2 | 16.3 ± 0.9% |
| Example 3 | 20.3 ± 1.8% |
| Example 4 | 16.3 ± 2.4% |

Referring to the left part of FIG. 8B and Table 6, the ABTS radical scavenging activity of the polymer dots of Examples 1 to 4 (not including the Preparation Example) is about 10% to about 30%, preferably about 15% to about 25%. In addition, as shown in the right part of FIG. 8B, the control group ABTS solution oxidized by potassium persulfate generates ABTS cation radicals, resulting in a stable bluish-green color and a maximum absorbance at 734 nm. The polymer dots of the examples are capable of scavenging the ABTS cation radicals, changing the solution color from bluish-green to transparent and colorless, and lowering the absorbance at 734 nm, indicating that the polymer dots of Examples 1 to 4 possess antioxidant activity.

The above results confirm that the polymer dots of the present disclosure exhibit excellent DPPH radical scavenging ability, capable of reducing oxidative stress on cells and tissues caused by excess radicals in the body, and are therefore useful for the preparation of antioxidant compositions.

The above disclosure merely illustrates preferred embodiments of the present invention. Equivalent modifications or variations made in accordance with the claims of the present invention shall fall within the scope of the invention.

What is claimed is:

1. A polymer dot, comprising a nitrogen-containing carbon dot that is free of benzene rings.

2. The polymer dot of claim 1, wherein a X-ray powder diffraction pattern of the nitrogen-containing carbon dot comprises characteristic peaks at 2θ values of 19.12°±0.2°, 19.44°±0.2°, 19.8°±0.2°, 20.04°±0.2°, and/or 20.28°±0.2°.

3. The polymer dot of claim 2, wherein the X-ray powder diffraction pattern of the nitrogen-containing carbon dot comprises a broad peak at a 2θ value of 42°±0.2°.

4. The polymer dot of claim 1, wherein a Zeta potential of the nitrogen-containing carbon dot is from −1 mV to 20 mV.

5. The polymer dot of claim 1, wherein the nitrogen-containing carbon dot is a dendritic carbon dot.

6. The polymer dot of claim 5, wherein the nitrogen-containing carbon dot has a non-conjugated structure.

7. The polymer dot of claim 1, wherein the nitrogen-containing carbon dot is formed from an A2-type monomer and a B3-type monomer, wherein the A2-type monomer is a dianhydride and the B3-type monomer is a polyetheramine.

8. The polymer dot of claim 7, wherein the nitrogen-containing carbon dot is formed by subjecting the A2-type monomer and the B3-type monomer to a one-pot synthesis.

9. The polymer dot of claim 8, wherein the nitrogen-containing carbon dot is formed by subjecting the A2-type monomer and the B3-type monomer to the one-pot synthesis and hydrothermal heating.

10. The polymer dot of claim 1, wherein the nitrogen content is from 5 to 10 atomic percent.

11. The polymer dot of claim 1, wherein the particle size of the nitrogen-containing carbon dot is greater than 10 nm.

12. The polymer dot of claim 1, wherein the nitrogen-containing carbon dot has an interplanar spacing of 4 Å to 6 Å corresponding to the (002) plane of graphite and/or the interplanar spacing of 1 Å to 3 Å corresponding to the (100) plane of graphite.

13. A method of preparing the polymer dot of claim 1, the method comprising: subjecting an A2-type monomer and a B3-type monomer by a one-pot synthesis to form the nitrogen-containing carbon dot.

14. The method of claim 13, further comprising: subjecting the A2-type monomer and B3-type monomer by a hydrothermal heating after the one-pot synthesis.

15. The method of claim 14, wherein the one-pot synthesis comprises: dissolving the A2-type monomer to form a first solution; dissolving the B3-type monomer to form a second solution; mixing the first solution and the second solution to form a mixed solution comprising a precipitate; and collecting and drying the precipitate; and wherein the hydrothermal heating comprises: diluting the precipitate to form a diluted solution; heating the diluted solution; collecting and filtering the supernatant of the diluted solution; and drying the supernatant.

16. The method of claim 15, wherein heating the diluted solution comprises: heating the diluted solution to a temperature of 150° C. to 250° C. under a pressure of 100 psi to 120 psi; and maintaining the temperature for 1 hour to 10 hours.

17. The method of claim 13, wherein the A2-type monomer is a dianhydride and the B3-type monomer is a polyetheramine.

18. Use of the polymer dot of claim 1 in a preparation of a composition for at least one of anti-inflammatory, antibacterial, and antioxidant purposes.
